(19) United States
(12) Patent Application Publication
(10) Pub. No.: US 2001/0011969 A1
(43) Pub. Date: Aug. 9, 2001

(54) Process and system for the generation of diagnostic-quality three-dimensional ultrasound image data sets

(76) Inventor: Hans Polz, Wartenberg (DE)

Correspondence Address: BIERMAN MUSERLIAN AND LUCAS, 600 THIRD AVENUE, NEW YORK, NY, 10016

(21) Appl. No.: 09/740,390

(22) Filed: Dec. 19, 2000

(30) Foreign Application Priority Data
Dec. 21, 1999 (DE) 199 61 651.5
Jan. 1, 2000 (DE) 100 01 817.3

(51) Int. Cl. G09G 1/14; A61B 5/05

(52) U.S. Cl. 345/23; 600/407

(57) ABSTRACT

The process and system generates three-dimensional ultrasound image data sets of diagnostic-quality. A probe which can be inserted into the patient is used to obtain the ultrasound images. The probe is used to generate position information along with the image or image data.

The invention pertains to a process according to preamble, patent claim 1 and to a system for implementation of this process according to preamble, patent claim 6.

A process for recording diagnostic-quality three-dimensional ultrasound image data sets is already known and described, e.g., in EP 0 865 765. An essential aspect of this known process is for example the free guidance of the ultrasound head during the recording of the individual ultrasound images, i.e. during the image or data acquisition.

For determining the position and orientation data in the known process, an electromagnetic sensor system is used, which is available, for example, under the designation “ISOTRAK II” from POLHEMUS, One Hercules Drive, Colchester, Vt. 05446.

The object of the invention is to demonstrate a process and a system for the generation of diagnostic-quality ultrasound image data sets in which the ultrasound images are provided by a probe, for example a TEE probe.

For the solution of this problem, a process corresponding to patent claim 1 is embodied. A system is embodied corresponding to patent claim 6.

Further embodiments of the invention are the subject of the dependent claims. The invention is described in more detail below using a sample embodiment in the figures, as follows:

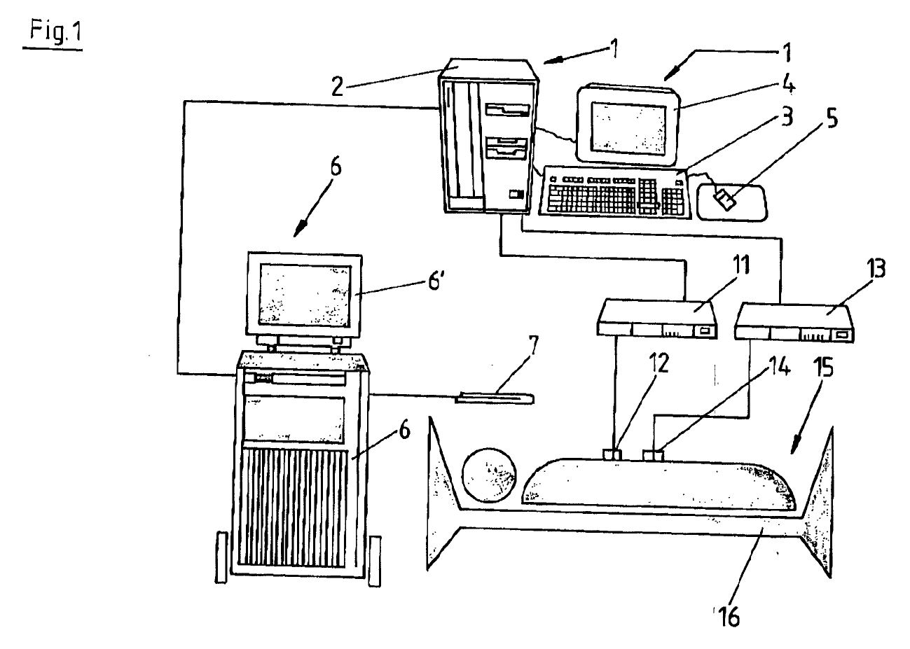

FIG. 1, a schematic representation of an embodiment of a system according to the invention for generating diagnostic-quality three-dimensional data sets for the tomographic imaging of a volume to be examined, using an insertable ultrasound probe;

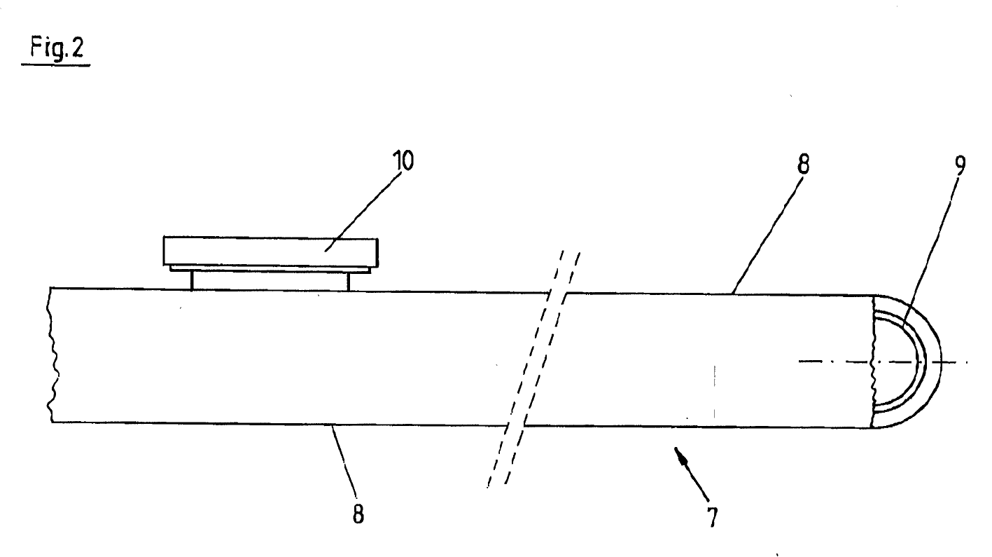

FIG. 2, a separate simplified representation of the probe

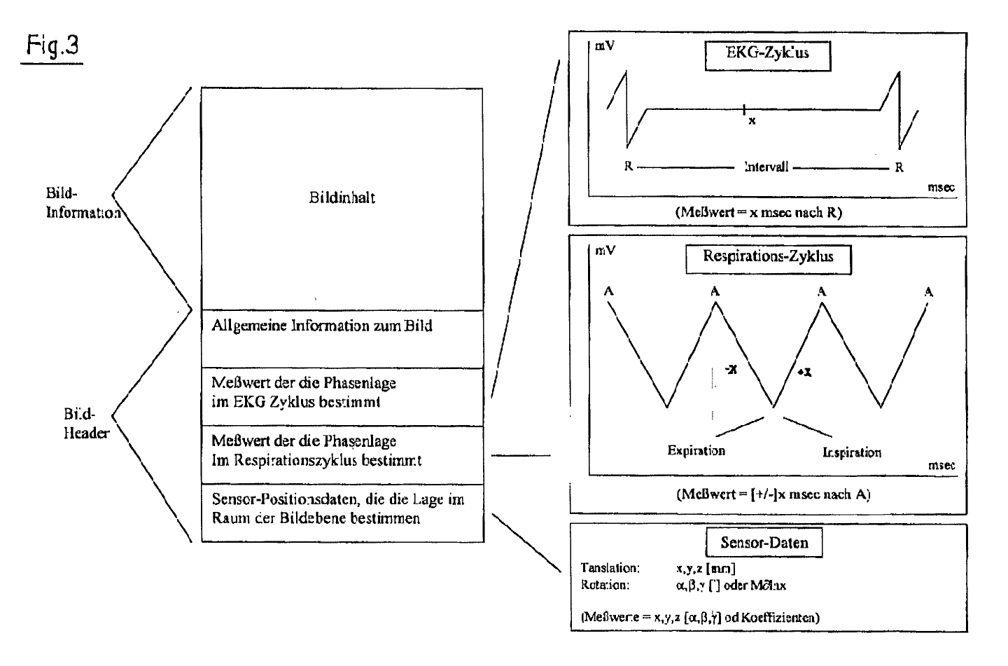

FIG. 3, a schematic representation of the single images stored in the image processing system, together with graphic representations of the EKG cycle and the respiration cycle of a patient.

The figures include 1 an image processing system, which essentially consists of the computer 2 and the peripheral equipment connected to it, such as keyboard 3, monitor 4, mouse control 5 etc.

Allocated to the image processing system 1 is an ultrasound device 6 with an ultrasound probe 7, which in the depicted embodiment is a TEE probe, i.e. a probe for trans-esophageal echocardiography.

Probes of this type are familiar to the expert. They consist essentially of a hose-type probe body 8 containing the functional elements of the probe and forming the tip of the probe on one end, on which within the probe body 8 an ultrasound crystal configuration 9 is located that is rotatable or swivelable on the probe axes, in a manner by means of a mechanism not depicted, for example via a flexible shaft within the probe body 8 by means of a gear or serve component 10. This is located on the probe body 8 at the necessary distance from the tip of the probe. The ultrasound configuration 9 provides images from image planes or sections that are radially oriented to the probe axis.

The probe 7 is connected to the ultrasound device 6 by means of a multiwire signal line, via which not only the image information can be transferred to the ultrasound device but also information defining the respective rotation position of the ultrasound head 9 provided by an electronic component within the probe 7, for example by an angle transmitter. The ultrasound device 6 generates from the image data provided by the probe 7 single ultrasound images whose image plane is dependent e.g. on the angle position of the ultrasound configuration 9. The single images can then be displayed on the monitor 6′ of the ultrasound device, together with information on the angle position of the ultrasound configuration 9 overlaid on the image.

In the depicted embodiment the image processing system is also allocated an EKG device 11 with corresponding electrodes 12 and a device 13 for registering the breathing (respiration cycle) with corresponding probes 14.

In order to produce a three-dimensional diagnostic-quality data set from the single images provided by the ultrasound device 6, the single images are recorded by the computer 2 of the image processing system 1 that is connected to a suitable output of the ultrasound device 6. If the single images are present in analog form, then suitable means, such as frame grabbers, are used to digitalize these analog images or video images. If the single ultrasound images are already present in digital form, then they can be recorded and processed by the computer without being converted. The image content of the single images also contains, as described above, the position information, i.e. the information on the angle position of the ultrasound configuration 9.

This position information is recognized and extracted using suitable software. In case analog single images are provided by the ultrasound device, this recognition and extraction of the position information takes place using a process or software for pattern recognition (e.g. OCR= optical character recognition).

In the computer, the respective position information for each single ultrasound image is stored in a data set that is allocated to this single image, corresponding to the image header of this data set in FIG. 3, whereby the image header in the depicted embodiment also contains values of the EKG device 11, e.g. a measured value that defines the phase position of the respective single image in relation to the EKG cycle, and/or e.g. a measured value of the device 13 that, for example, defines the phase position of the single image in relation to the respiration cycle.

The probe 7, which for example enables the recording of image sequences in various sectional planes of the human heart, is inserted into the esophagus of the patient 15.

The allocation of the position information to the respective ultrasound image, i.e, the storing of this information and additional information in the image header can take place online, i.e. simultaneously with the recording of the single images or also after the image recording in the first step of subsequent processing.

Especially if the position information is processed “online”, this information can be used to control the image recording or the image feeding by the computer 2, i.e. a single ultrasound image is only recorded if a pre-defined angle increment of the rotation position of the ultrasound configuration 9 has been traveled since the last recorded single image.

The EKG device and the device 13 make it possible to generate dynamic or static three-dimensional data sets, with respect to the heart cycle or the respiration of the patient 15. In particular, this also enables EKG triggering and respiratory gating.

The image data stored in the computer 2 with the corresponding image headers form a rough data set from which then, for example by a transformation or interpolation process, the diagnostic-quality three-dimensional data set is created in a standard reference or coordinate system. To do this, the method described in detail in EP 0 865 765 is followed, for example.

If in addition to the actual position information that is provided and displayed by the ultrasound device or the corresponding probe, geometric assumptions are made, e.g. rotation of the ultrasound configuration 9 on an axis defined in its position, for example on an axis extending through the middle of the ultrasound head 9, then these assumptions are also stored in the respective image header, so that this data can be included in the generation of the three-dimensional data set.

The position information provided by the probe 7, i.e. information on the angle position of the ultrasound configuration 9 is for example numeric and/or graphic information.

The invention was described above using a sample embodiment. Of course, modifications and adaptations are possible without abandoning the inventive idea on which the invention is based.

If the single images provided by the ultrasound device 6 already contain the position information in the image header, then this information can be used directly for the creation of the three-dimensional volume data set.

Furthermore, it is possible that the computer 2 is part of the ultrasound device 6. In this case it is possible that the corresponding program for generating the ultrasound images from the ultrasound signals of the probe 7 and the position information transfer this image data and position information via a suitable interface. e.g. via OLE interface or shared memory file or shared memory mechanisms to the program for generating the three dimensional volume data set.

1. Process for generating a diagnostic-quality three-dimensional image data set using an ultrasound device with an ultrasound configuration (9) for producing a sequence of ultrasound images in various planes of the volume to be examined, using an image processing facility (1) to which the single ultrasound images are transferred and using means of producing position information defining the position of the ultrasound configuration (9) that is also transferred to the image processing facility (1), which generates a three-dimensional volume data set for tomographic recording of the examined volume from the image data of the single ultrasound images and the position information, characterized in that the at least one ultrasound configuration (9) is part of a probe (7), preferably part of a TEE probe.

2. Process according to claim 1, characterized in that the at least one ultrasound configuration (9) is part of a probe (7) that can be inserted into the volume to be examined.

3. Process according to claim 1 or 2, characterized in that the ultrasound device generates single images from the image data provided by the probe (7) containing the position information in the respective image or in the respective image data, and that this information is extracted in the image processing facility and is stored together with the image data, preferably in an image header of the image data.

4. Process according to one of the foregoing claims, characterized in that the position information is taken over by the data processing facility (1) from an image header of the single images created by the ultrasound device (6).

5. Process according to one of the foregoing claims, characterized in that in case the image processing facility (1) is integrated into the ultrasound device (6) the position information determined by a computer from the data of the probe (7) is taken over directly for generating the three-dimensional image data set.

6. System for generating a diagnostic-quality three-dimensional image data set using an ultrasound device with an ultrasound configuration (9) for producing a sequence of ultrasound images in various planes of the volume to be examined, using an image processing facility (1) to which the single ultrasound images are transferred and using means of producing position information defining the position of the ultrasound configuration (9) that is also transferred to the image processing facility (1), which generates a three-dimensional volume data of the for tomographic recording of the examined volume from the image data of the single ultrasound images and the position information, characterized in that the at least one ultrasound configuration (9) is part of a probe (7) that can be inserted into the volume to be examined, preferably part of a TEE probe.

7. System according to claim 6, characterized in that the at least one ultrasound configuration (9) is put of a probe (7) that can be inserted into the volume to be examined.

8. System according to claim 6 or 7, characterized in that the ultrasound device generates single images from the image data provided by the probe (7), which contains the position information in the respective image or in the respective image data, and that this information is extracted in the image processing facility (1) and stored together with the image data preferably in an image header of the image data.

9. System according to one of the foregoing claims, characterized in that the position information is taken over by the data processing facility (1) from an image header of the single images created by the ultrasound device (6).

10. System according to one of the foregoing claims, characterized in that in case the image processing facility (1) is integrated in the ultrasound device (6), the position information determined by a computer from the data of the probe (7) is taken over directly for generating the three-dimensional image data set.
